(19) United States
(12) Patent Application Publication
(10) Pub. No.: US 2003/0206972 A1
(43) Pub. Date: Nov. 6, 2003

(54) Compositions containing carotenoids and tocotrienols and having synergistic antioxidant effect

(76) Inventors: John G. Babish, Brooktondale, NY (US); Terrence Howell, Dryden, NY (US)

Correspondence Address: THORPE NORTH & WESTERN, L.L.P., Attn: M. Wayne Western, P.O. Box 1219, Sandy, UT, 84091-1219, US

(21) Appl. No.: 09/976,255

(22) Filed: Oct. 11, 2001

Related U.S. Application Data
(60) Provisional application No. 60/240,208, filed on Oct. 13, 2000.

(51) Int. Cl. A61K 35/78; A61K 31/355; A61K 31/12; A61K 31/015

(52) U.S. Cl. 424/725; 514/458; 514/683; 514/763

(57) ABSTRACT

A novel formulation is provided that serves to synergistically inhibit the generation of free radicals and oxidative stress in warm blooded animals. The formulation comprises an effective amount of a first component of a carotenoid species, and, as a second component, a tocotrienol species and derivatives thereof, and provides for synergistic anti-oxidant activity.

### CROSS REFERENCE TO RELATED APPLICATIONS

This application claims benefit of U.S. Provisional Application Serial No. 60/240,208 filed Oct. 13, 2000.

### FIELD OF THE INVENTION

The present invention relates generally to a composition exhibiting synergistic antioxidant activity. More particularly, the composition comprises, as a first component, a carotenoid species, and, as a second component, a tocotrienol species or derivatives thereof. The composition exhibits synergistic antioxidant activity.

### BACKGROUND OF THE INVENTION

Oxygen is essential for aerobic life, but is also a precursor to the formation of harmful reactive oxygen species (ROS). Oxidative stress refers to the cytotoxic consequences of a mismatch between the production of free radicals and the ability of the cell to defend against them. Oxidative stress can thus occur when the formation of ROS increases, scavenging of ROS or repair of oxy-modified macromolecules decreases, or both. ROS may be oxygen-centered radicals possessing unpaired electrons, such as superoxide and hydroxyl radicals, or covalent molecules, such as hydrogen peroxide.

Superoxide and hydrogen peroxide are relatively nonreactive toward biological molecules. Hydroxyl radicals, on the other hand, are highly reactive. Under physiological conditions, superoxide is converted to hydrogen peroxide by the enzyme superoxide dismutase (SOD) or by interaction with transition metals. Hydrogen peroxide is in turn reduced to water by glutathione peroxidase or converted to oxygen and water by catalase. Thus, the hydroxyl radical represents the greatest threat to cell viability.

ROS, especially hydroxyl radicals, can produce functional alterations in lipids, proteins, and nucleic acids. The incorporation of molecular oxygen into polyunsaturated fatty acids initiates a chain reaction in which ROS, including hydroxyl radicals, hydrogen peroxide, and peroxyl and alkoxyl radicals are formed. Oxidative lipid damage, termed lipid peroxidation, results in a progressive loss of membrane fluidity, reduces membrane potential, and increases permeability to ions such as calcium. ROS can damage proteins and change amino groups on amino acids resulting in the inactivation of the proteins. DNA and RNA are also targets of ROS. Hydroxyl radicals modify ribose phosphates, pyrimidine nucleotides and nucleosides and react with the sugar phosphate backbone of DNA causing breaks in the DNA strand.

Because ROS and the associated oxidative stress can produce fundamental cellular damage, primary or secondary oxidative insults have been implicated in many diseases. Table 1 below provides a list of physiological insults in which oxidative stress and ROS are believed to play a significant role and are therefore appropriate targets for normalization, prevention or treatment by antioxidants.

TABLE 1

| Physiological Insults Generating | |
|---|---|
| Oxidative Stress | Affected Tissues or Systems |
| Addison's Disease | Adrenal |
| Aging | Skin and other systems |
| Allergies | Inflammatory cells |
| Alzheimer Disease | Nerve cells |
| Angioplasty | Arterial epithelial cells |
| Arthritis | Inflammatory cells |
| Asthma | Immune cells |
| Atherosclerosis | Vessel wall |
| Cigarette Smoking | Lung, mouth, throat and blood vessels |
| Colon Cancer | Intestine |
| Chacaxia | Muscular and Nervous |
| Crohn's Disease | Intestine |
| Cystic Fibrosis | Lungs |
| Diabetes (type I and type II) | Pancreas and various systems |
| Eczema | Skin/Inflammatory cells |
| Exercise | muscle, liver and fat |
| Graves' Disease | Thyroid |
| Guillain-Barre Syndrome | Nerve cells |
| Head Injury | Brain |
| Hemodialysis | Kidney |
| Hepatitis | Liver |
| HIV-1 Infection | Muscular and Immune systems |
| Hypercholesterolemia | Arterial vessels |
| Hyperlipidemia | Liver and Arterial vessels |
| Hyperthyroidism | Thyroid |
| Inflammation | Immune cells |
| Inflammatory Bowel Disease | Intestine |
| Leukemia | Immune cells |
| Lymphomas | Immune cells |
| Multiple Sclerosis | Nerve cells |
| Myasthenia Gravis | Neuromuscular junction |
| Nuclear Factor kappaB Activa- | Immune cells |
| tion | |
| Neurodegeneration | Central nervous system |
| Physical Exertion | Muscular and Immune systems |
| Psoriasis | Skin |
| Primary Biliary Cirrhosis | Liver |
| Reperfusion Injury | Head and heart |
| Rheumatoid Arthritis | Joint lining |
| Solid Tumors | Various |
| Systemic Lupus Erythematosis | Multiple tissues |
| Tumor Necrosis Factor-alpha | Various Systems |
| Expression | |
| Uveitis | Eye Various Systems |
| Weight loss | Muscle |

Numerous epidemiological investigations have suggested that consumption of antioxidants in the form of fresh fruits and vegetables provides protection from cancer, cardiovascular disease, autoimmune disease and neurodegeneration. Furthermore, in vitro studies support the palliative effects of single, purified antioxidant treatment in a variety of model systems. In particular, carotenoids have been a focus of study with respect to decreasing oxidative stress as well as cancer prevention and intervention.

Carotenoids (FIG. 1[A]) are a family of over 700 natural, lipid-soluble pigments that are only produced by phytoplankton, algae, plants and a limited number of fungi and bacteria. The carotenoids are responsible for the wide variety of colors they provide in nature, most conspicuously in the yellow and red colors of fruits and leaves. In plants and algae, carotenoids along with chlorophyll and other light-harvesting pigments are vital participants in the photosynthetic process.

Biologically, carotenoids are distinguished by their capacity to interact with singlet oxygen and free radicals. Among the carotenoids, a growing body of scientific literature describes astaxanthin as one of the best antioxidants. Due to its unique molecular structure among carotenoids (a carbonyl and hydroxyl group on each of the terminal aromatic rings), astaxanthin has both a potent quenching effect against singlet state oxygen and a powerful scavenging ability for free radicals. Thus, astaxanthin serves as an extremely effective antioxidant against these reactive species. However, experience with cancer intervention trials has shown that supplementation with a single antioxidant may produce untoward, stimulatory effects on cancer growth.

Numerous epidemiological investigations have shown that cancer risk is inversely related to the consumption of green and yellow vegetables and fruits. Since beta-carotene is present in abundance in these vegetables and fruits, it has been investigated extensively in the laboratory as a possible cancer-preventive agent. In addition, other carotenoids also have been investigated for their antioxidant and anti-carcinogenic activity. For example, lutein, zeaxanthin, lycopene, phytoene, fucoxanthin, peridinin and astaxanthin seem to be promising. Among these later carotenoids, astaxanthin has most recently demonstrated the greatest antioxidant activity.

However, as a result of clinical studies, the role of carotenoids as anticancer supplements has recently been questioned. For example, the incidence of non-melanoma skin cancer was unchanged in patients receiving a β-carotene supplement. A recent study also demonstrated that smokers gained no benefit from supplemental β-carotene with respect to lung cancer incidence and possibly even suffered a deleterious effect. This inference also extends to numerous other diseases associated with oxidative stress such as Alzheimer's disease, diabetes and cardiovascular disease (Table 1). Also see, Hennekens, et al. Lack of effect of long-term supplementation with β-carotene on the incidence of malignant neoplasms and cardiovascular disease. N. Engl. J. Med. 334:1145-1149 (1996); Greenberug, et al, A clinical trial of β-carotene to prevent basal cell and squamous cell cancers of the skin. N. Engl. J. Med. 323:789-795 (1990).

Consequently, it has been inferred that multiple antioxidant supplementation would be necessary to achieve clinical effectiveness. The most rational approach for the demonstration of antioxidant combinations is the identification of synergy between the components of the formulation. Such a synergistic combination would theoretically increase the likelihood of a positive clinical outcome. Therefore, it would be useful to produce a potent combination of antioxidants that function synergistically to inhibit the generation of free radicals and thus positively affect the initiation, progression and pathology of the targeted disease.

Tocotrienols are a family of dietary supplements related to vitamin E and are considered to be powerful antioxidants. Although they can be chemically synthesized, the best natural sources for tocotrienols are the oils derived from rice bran, palm fruit, barley and wheat germ. Comparatively, the tocotrienol structure differs from the tocopherol structure by possessing three double bonds in its side chain rather than being saturated as is the tocopherol [FIG. 2].

Tocotrienols have been shown to elicit powerful antioxidant, anti-cancer and cholesterol-lowering properties and such physiological properties appear to be much stronger than tocopherols. See U.S. Pat. Nos. 4,603,141; 5,217,992; 5,348,974; and 5,393,776; 5,591,772; and 5,919,818. Overall, it has been concluded that the transport, tissue concentration profile, and relative biologic function of the tocopherols and tocotrienols appear somewhat disparate and possibly unrelated. Because they lack vitamin E activity, the tocotrienols were once thought to be of lesser nutritional value than the tocopherols. From their antioxidant activity, however, they may become one of the most important nutritional compounds for the prevention and treatment of disease. See U.S. Pat. Nos. 5,545,398 and 5,709,868.

Therefore, it would be useful to provide formulations of compounds that would function synergistically with a carotenoid or tocotrienol species to increase the antioxidant activity of both species in excess of their individual contribution.

### SUMMARY OF THE INVENTION

The present invention provides a composition having a synergistic inhibitory effect on biological oxidative processes involving free radicals or singlet oxygen. The present invention provides a composition comprising, as a first component, a carotenoid species, and, as a second component, a tocotrienol species or derivatives thereof. The composition exhibits synergistic antioxidant activity.

Preferably, the carotenoid species is a member selected from the group consisting of astaxanthin, alpha-carotene, beta-carotene, lutein, lycopene, zeaxanthin, phytoene, fucoxanthin, peridin,and cantaxanthin. More preferably, the carotenoid species is a member selected from the group consisting of astaxanthin, beta-carotene, lutein, and lycopene. The most preferred carotenoid species is astaxanthin.

Preferably, the tocotrienol species is a member selected from the group consisting of tocotrienol, alpha-, beta-, gamma-, delta-tocotrienol, desmethyl-tocotrienol, didesmethyl-tocotrienol, and mixtures thereof. More preferably the tocotrienol species is a member selected from the group of tocotrienol, alpha-, beta-, gamma-, delta-tocotrienol and mixtures thereof. The most preferred composition of the tocotrienol species is a mixture of alpha- and beta-tocotrienol or a mixture of alpha-, beta-, gamma- and delta-tocotrienol. The composition functions synergistically to inhibit the generation of free radicals and oxidative stress.

The present invention also provides a composition of matter which enhances the normal functioning of the body in times of oxidative stress resulting from a chronic debilitating disease.

The present invention further provides a method of dietary supplementation and a method of treating oxidative stress or oxidative stress-based diseases in a warm-blooded animal which comprises providing to the animal suffering symptoms of oxidative stress the composition of the present invention containing a second component which specifically and synergistically enhances the antioxidant activity of carotenoid species and/or tocotrienol species and continuing to administer such a dietary supplementation of the composition until said symptoms are eliminated or reduced.

### BRIEF DESCRIPTION OF THE DRAWINGS

FIGS. 1[A] and [B] respectively, illustrate the general chemical structure of the carotenoid genus and astaxanthin (3,3′-dihydroxy-β,β-carotene-4,4′-dieto-β-carotene) as a species within that genus.

FIG. 2[A] represents the general structure of tocopherols, when R₁ and R₂=—CH₃ it represents alpha-tocopherol; when R₁=—CH₃ and R₂=H it represents beta-tocopherol; when R₁=H and R₂=—CH₃ it represents gamma-tocopherol and when R₁ and R₂=H it represents delta-tocopherol; 2[B] represents the general structure of tocotrienols, when R₁ and R₂=—CH₃ it represents alpha-tocotrienol; when R₁=—CH₃ and R₂=H it represents beta-tocotrienol; when R₁=H and R₂=—CH₃ it represents gamma-tocotrienol and when R₁ and R₂=H it represents delta-tocotrienol; 2[C] is the structure of trolox (6-hydroxy-2,5,7,8-tetramethylchroman-2-carboxylic acid, the minimum structure exhibiting vitamin E activity.

### DETAILED DESCRIPTION OF THE INVENTION

Before the present composition and methods of making and using thereof are disclosed and described, it is to be understood that this invention is not limited to the particular configurations, as process steps, and materials may vary somewhat. It is also intended to be understood that the terminology employed herein is used for the purpose of describing particular embodiments only and is not intended to be limiting since the scope of the present invention will be limited only by the appended claims and equivalents thereof.

It must be noted that, as used in this specification and the appended claims, the singular forms “a,” “an,” and “the” include plural referents unless the context clearly dictates otherwise.

The present invention provides a composition having a synergistic antioxidant activity. More particularly, the composition comprises, as a first component, at least one member of the carotenoid species, and, as a second component, at least one member of the tocotrienol species or derivatives thereof. Preferably, the molar ratio of the first component, i.e. the carotenoid species, to the second component, i.e. tocotrienol species or derivatives thereof, is within a range of 500:1 to 1:12,000. The composition provided by the present invention can be formulated as a dietary supplement or therapeutic composition. The composition functions synergistically to inhibit biological oxidation involving free radicals or singlet oxygen. Such combinations are useful as dietary supplements or therapeutics for the physiological insults listed in Table 1.

As used herein, the term “dietary supplement” refers to compositions consumed to affect structural or functional changes in physiology. The term “therapeutic composition” refers to any compounds administered to treat or prevent a disease.

As used herein, the term “antioxidant activity” refers to an inhibitory effect on biological oxidative processes involving free radicals or singlet oxygen.

As used herein, carotenoid species, tocotrienol species and derivatives thereof are meant to include naturally occurring or synthetic derivatives of species within the scope of the respective genera. Natural derivatives may be obtained from common microbiological or plant sources and may exist as conjugates.

“Conjugates” of carotenoid species, tocotrienol species and derivatives thereof means carotenoid species, tocotrenol, alpha-, beta-, gamma- and delta-tocotrienol or derivatives thereof covalently bound or conjugated to a member selected from the group consisting of mono- or di-saccharides, amino acids, fatty acids, sulfates, succinate, acetate and glutathione. Preferably, the fatty acid is a C₆ to C₂₂ fatty acid. Preferably, the mono- or di-saccharide is a member selected from the group consisting of glucose, mannose, ribose, galactose, rhamnose, arabinose, maltose and fructose.

Preferably, the carotenoid species is a member selected from the group consisting of astaxanthin, alpha-carotene, beta-carotene, lutein, lycopene, zeaxanthin, phytoene, fucoxanthin, peridin,and cantaxanthin. More preferably, the carotenoid species is a member selected from the group consisting of astaxanthin, beta-carotene, lutein, and lycopene. The most preferred carotenoid species is astaxanthin.

The tocotrienols of the present invention including, but not limited to, both natural tocotrienol, alpha-, beta-, gamma-, delta-tocotrienol, desmethyl-tocotrienol, and didesmethyl-tocotrienol as well as synthetic derivatives or conjugates, and mixtures thereof. Preferably, the tocotrienol species is a member selected from the group consisting of tocotrienol, alpha-, beta-, gamma-, delta-tocotrienol, desmethyl-tocotrienol, didesmethyl-tocotrienol, and mixtures thereof. More preferably the tocotrienol species is a member selected from the group of tocotrenol, alpha-, beta-, gamma-, delta-tocotrienol and mixtures thereof. The most preferred composition of the tocotrienol species is a mixture of alpha- and beta-tocotrienol or a mixture of alpha-, beta-, gamma- and delta-tocotrienol.

Therefore, one preferred embodiment of the present invention is a composition comprising a combination of an effective amount of astaxanthin as a first component, and, as a second component, at least one member selected from the group consisting of tocotrienol, alpha-, beta-, gamma-, or delta-tocotrienol and derivatives thereof. The resulting formulation of these combinations exhibits synergistic antioxidant activity.

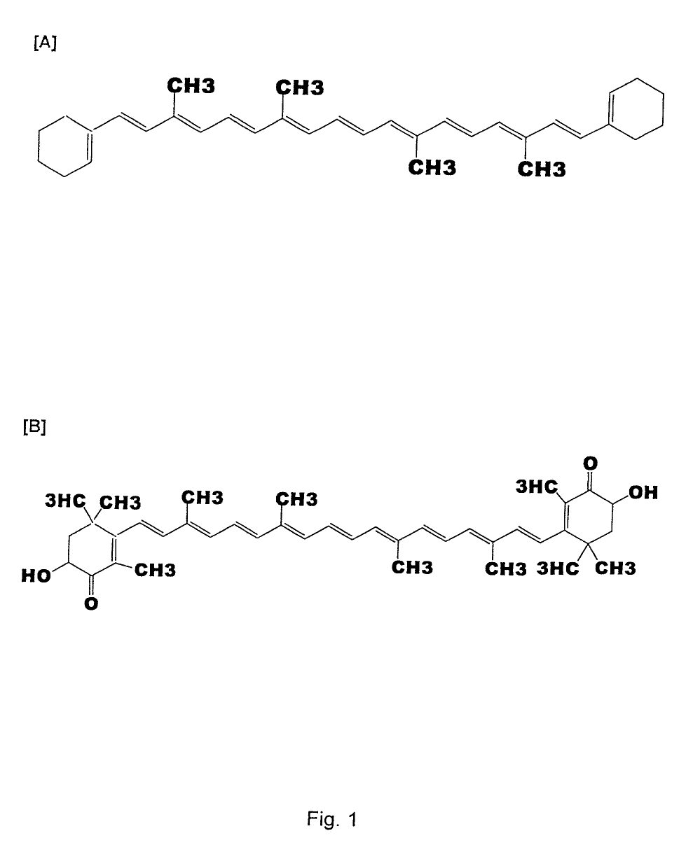

Preferably, the carotenoid or astaxanthin (FIGS. 1[A] and [B], respectively) employed in the present invention is a pharmaceutical grade preparation such as can be obtained commercially, for example, from Reisman Corporation, Orange. The pharmaceutical grade extract must pass extensive safety and efficacy procedures. Pharmaceutical grade astaxanthin is standardized to have a greater than one weight percent of astaxanthin and can be readily obtained from the green algae Haematococcus pluvialis. As employed in the practice of the invention, the astaxanthin extract has an astaxanthin content of about 1.0 to 95 percent by weight. Preferably, the minimum astaxanthin content is about 2 percent by weight. Alternatively, the astaxanthin maybe synthesized using standard techniques known in chemical synthesis.

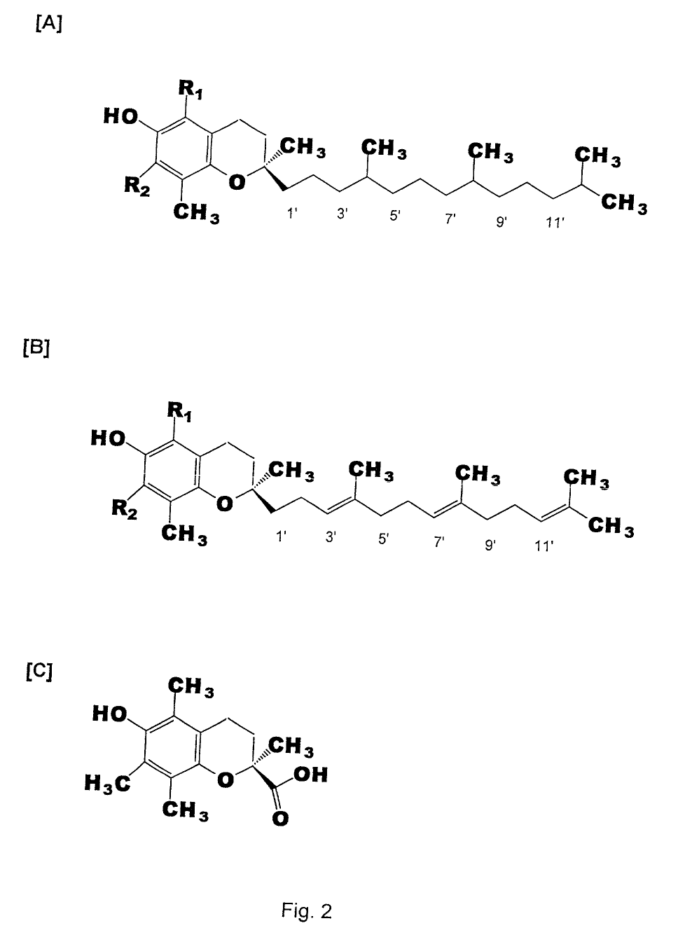

The preferred tocotrienol employed (FIG. 2[B]) is a pharmaceutical grade preparation that can be obtained from Eastman Kodak, Rochester, N.Y. In general, alpha-, beta-, gamma- and delta-tocotrienol are obtained in the form of standardized mixtures of the oil derived from rice bran, palm fruit, barley and wheat germ. Pharmaceutical grade tocotrienols contain at least 7 percent by weight of tocotrienols. As employed in the practice of this invention the oily tocotrienol extracts contain a minimum tocotrienol content of 1 to 50 percent by weight.

The botanical sources for tocotrienol species includes, but not limited to, wheat germ, barley, palm fruit, rice bran, sunflower seeds, vegetable oils, brewer's grains, oats, and African violets. The preferred botanical sources for alpha-, beta-, gamma- and delta-tocotrienol is the lipid fraction selected from the group consisting of wheat germ, barley, palm fruit and rice bran. The most preferred botanical source for alpha, beta, gamma and delta-tocotrienol is the lipid fraction selected from the group consisting of palm fruit and rice bran.

Without limiting the invention, the action of the second component of the composition is thought to provide a dual, synergistic antioxidant effect with the first component. The second compound can also provide hepatoprotection, antitumor promotion, antihyperlipidemia, and antihyperglycemia.

A daily dose (mg/day) of the present dietary supplement would be formulated to deliver: 0.1 to 50 mg of the first component, i.e. a carotenoid species, and 0.5 to 2500 mg of the second component, i.e. alpha-, beta-, gamma- or delta-tocotrienol or a mixture or derivative thereof.

Preferably, the daily dose (mg/day) of the present dietary supplement would be formulated to deliver: 3 to 15 mg of the first component, i.e. carotenoid species, and 30 to 600 mg of the second component, i.e. alpha-, beta-, gamma- or delta-tocotrienol or a mixture or derivative thereof.

The composition of the present invention for topical application would contain 0.001 to 10 weight percent, preferably 0.05 to 2 weight percent, of the first component, i.e. a carotenoid species, and, 0.001 to 10 weight percent, preferably 0.05 to 2 weight percent, of the second component, i.e. alpha-, beta-, gamma- and delta-tocotrienol and derivatives thereof.

The preferred composition of the present invention would produce serum or tissue concentrations in the following range: 0.01 to 5,500 μM of the first component, i.e. carotenoid species, and 0.001 to 50 μM of the second component, i.e. alpha-, beta-, gamma- or delta-tocotrienol.

In addition to the combination of active ingredients selected from the group consisting of a carotenoid species, alpha-, beta-, gamma- and delta-tocotrienol and derivatives thereof, the present composition for dietary application may include various additives such as other natural components of intermediary metabolism, vitamins and minerals, as well as inert ingredients such as talc and magnesium stearate that are standard excipients in the manufacture of tablets and capsules.

As used herein, “pharmaceutically acceptable carrier” includes any and all solvents, dispersion media, coatings, isotonic and absorption delaying agents, sweeteners and the like. These pharmaceutically acceptable carriers may be prepared from a wide range of materials including, but not limited to, diluents, binders and adhesives, lubricants, disintegrants, coloring agents, bulking agents, flavoring agents, sweetening agents and miscellaneous materials such as buffers and absorbents that may be needed to prepare a particular therapeutic composition. The use of such media and agents for pharmaceutically active substances is well known in the art. Except insofar as any conventional media or agent is incompatible with the active ingredients, its use in the present composition is contemplated. In one embodiment, talc and magnesium stearate are included in the present formulation. Other ingredients known to affect the manufacture of this composition as a dietary bar or functional food can include flavorings, sugars, amino-sugars, proteins and/or modified starches, as well as fats and oils.

The dietary supplements, lotions or therapeutic compositions of the present invention can be formulated in any manner known by one of skill in the art. In one embodiment, the composition is formulated into a capsule or tablet using techniques available to one of skill in the art. In capsule or tablet form, the recommended daily dose for an adult human or animal would preferably be contained in one to six capsules or tablets. However, the present compositions may also be formulated in other convenient forms such as, an injectable solution or suspension, a spray solution or suspension, a lotion, gum, lozenge, food or snack item. Food, snack, gum or lozenge items can include any ingestible ingredient, including sweeteners, flavorings, oils, starches, proteins, fruits or fruit extracts, vegetables or vegetable extracts, grains, animal fats or proteins. Thus, the present composition can be formulated into cereals, snack items such as chips, bars, gumdrops, chewable candies or slowly dissolving lozenges.

The present invention contemplates treatment of all types of oxidative stress-based diseases, both acute and chronic. The present formulation reduces the symptoms of oxidative stress and thereby promotes healing of, or prevents further damage to, the affected tissue. A pharmaceutically acceptable carrier may also be used in the present compositions and formulations.

According to the present invention, the warm blooded animal may be a member selected from the group consisting of humans, non-human primates, such as dogs, cats, birds, horses, ruminants or other warm blooded animals. The invention is directed primarily to the treatment of human beings. Administration can be by any method available to the skilled artisan, for example, by oral, topical, transdermal, transmucosal, or parenteral routes.

The following examples are intended to illustrate but not in anyway limit the invention.

### EXAMPLE 1

### Antioxidant Synergy Exhibited for the Combination of Astaxanthin and Mixed Tocotrienols

This example illustrates the antioxidant synergy between astaxanthin and a mixture of tocotrienols.

Measurement of peroxy radical scavenging activity of the individual compounds and combinations was performed essentially as described by Naguib (Analytical Biochemistry (1998) 265:290-298) with modifications to allow the assay to be performed on a microplate. The fatty acid indicator 4,4-difluoro-5-(4-phenuyl-1,3-butadienyl)-4-bora-3a,4a-diaza-s-indacene-3-undecanate (BODIPY 581/591 C₁₁) was purchased from Molecular Probes (Eugene, Oreg.). As a peroxy radical generator, 2,2′-azobis-2,4-dimethyl valeronitrile (AMVN) was used and obtained from WAKO Chemicals (Richmond, Va.). All standards were of the highest purity commercially available. Additionally, the solvents were of HPLC grade.

Fluorescence measurements were performed using a Packard FluoroCount microplate fluorometer equipped with a temperature-controlled plate holder. All measurements were preformed in a 96-well polypropylene plate with stirring. The fluorescence signal of the indicator BODIPY 581/591 C₁₁ in octane:butyronitrile (9:1, v/v) at 30° C. gradually decreased upon addition of the peroxyl radical-generating system AMVN.

Reactions in octane:butyronitrile (9:1, v/v) were carried out at 30° C. All fluorescence measurements were recorded every 15 minutes for 1 hour at excitation wavelengths of 570 nm, with emission at 620 nm. The net protection (area under the curve, AUC) provided by an antioxidant sample was calculated using the trapezoid method. Percent inhibition was calculated based upon AUC_{(AMVN)}=0% inhibition and AUC_{(BODIPY)}=100% inhibition. The dynamic range of inhibition was defined as the AUC between 0 and 100%=AUC_{(BODIPY)}−AUC_{(AMVN)}. The percent inhibition of each dose was calculated as:

[AUC_{(dose)}−AUC_{(AMVN)}]/[AUC_{(BODIPY)}−AUC_{(AMVN)}]×100.

The final reaction mixture (200 μL) for the assay contained 0.5 μg/mL BODIPY 581/591 C₁₁ and 0.26 M AMVN in octane:butyronitrile (9:1, v/v). Stock solutions of the test samples were made up in chloroform and 6 μL were added to the reaction mixture to achieve the stated concentrations. The reaction was initiated by the addition of the AMVN and fluorometric readings were recorded every 15 minutes for one hour.

Synergy between astaxanthin and the sample of mixed tocotrienols was assessed using CalcuSyn (BIOSOFT, biosoft.com). This statistical package performs multiple drug dose-effect calculations using the Median Effect methods described by T-C Chou and P. Talaly (Trends Pharmacol. Sci. 4:450-454), hereby incorporated by reference.

Briefly, it correlates the “Dose” and the “Effect” in the simplest possible form: fa/fu=(C/Cm)m, where C is the concentration or dose of the compound and Cm is the median-effective dose signifying the potency. Cm is determined from the x-intercept of the median-effect plot. The fraction affected by the concentration of the test material is fa and the fraction unaffected by the concentration is fu (fu=1−fa). The exponent m is the parameter signifying the sigmoidicity or shape of the dose-effect curve. It is estimated by the slope of the median-effect plot.

The median-effect plot is a plot of x=log(C) vs y=log(fa/fu) and is based on the logarithmic form of Chou's median-effect equation. The goodness of fit for the data to the median-effect equation is represented by the linear correlation coefficient r of the median-effect plot. Usually, the experimental data from enzyme or receptor systems have an r>0.96, from tissue culture an r>0.90 and from animal systems an r>0.85.

Synergy of test components is quantified using the combination index (CI) parameter. The CI of Chou-Talaly is based on the multiple drug-effect and is derived from enzyme kinetic models (Chou, T.-C. and Talalay, P. (1977) A simple generalized equation for the analysis of multiple inhibitions of Michaelis-Menten kinetic systems. J. Biol. Chem. 252:6438-6442). The equation determines only the additive effect rather than synergism or antagonism. However, we define synergism as a more than expected additive effect, and antagonism as a less than expected additive effect as proposed by Cho and Talalay in 1983 (Trends Pharmacol. Sci. (1983) 4:450-454). Using the designation of CI=1 as the additive effect, we obtain for mutually exclusive compounds that have the same mode of action or for mutually non-exclusive drugs that have totally independent modes of action the following relationships: CI<1, =1, and >1 indicate synergism, additivity and antagonism, respectively.

The astaxanthin was obtained from Sigma (A9335; St. Louis, Mo.) and the 50 percent oil mixture of tocotrienols was obtained from H. Reisman Corp (Lot no. B1005-1-080799; Orange, N.J.). Within the tocotrienol fraction, approximately 56% was reported asd-gamma-tocotrienol, 30% as d-alpha-tocotrienol, 13% as d-delta-tocotrienol and 1% other tocotrienols including d-beta-tocotrienol. Dose-response curves were described with each test article separately and then in a two-way combination. For the individual dose-response curves, concentrations of astaxanthin included 24, 48, 96 and 192 μg/mL; the mixed tocotrienol concentrations were 11, 22, 44, 88, and 176 μg/mL. Following the estimation of the component IC50 values, serial dilutions of a mixture containing concentrations of each component equal to 4 times the IC50 value were assayed. This resulted in a series of test concentrations of the mixture containing 4, 2, 1, 0.5, and 0.25 times the IC50 concentration of each component.

Table 1.1 depicts the calculated inhibitory concentrations and combination indexes for 50, 75 and 90 percent inhibition of peroxy radical formation by the combination of astaxanthin with mixed tocotrienols. When the mixture response was parsed into the astaxanthin and tocotrienol components, values of 66, 135 and 230 μg/mL, respectively, were estimated for 50, 75 and 90 percent inhibition by astaxanthin. Similarly, the tocotrienol concentrations for the same three parameters were 66, 125 and 230 μg/mL. The calculated combination index for each of the values indicated synergy over the complete dose-response curve.

TABLE 1.1
Statistical results of the antioxidant effect of a combination of
astaxanthin and a 50 percent solution of mixed tocotrienols

| | IC50* | IC75* | IC90* |
|---|---|---|---|
| Test Material | [μg/mL] | [μg/mL] | [μg/mL] |
| Astaxanthin alone | 150 (115-194) | | |
| 50% Tocotrienols | 67 (60-75) | | |
| alone | | | |
| Mixture - | 66 (59-77) | 125 (107-145) | 230 (190-279) |
| Astaxanthin | | | |
| Mixture - | 31 (27-36) | 57 | 106 |
| Tocotrienols | | | |
| Combination | 0.92 | 0.91 | 0.92 |
| Index = | | | |

*Exhibited significant (p < 0.5) synergy with CI < 1.0; parenthetic values are 95% confidence intervals for the estimated value.

This combination of astaxanthin and mixed tocotrienols effectively increased the antioxidant potency of astaxanthin 2.3-fold; astaxanthin alone exhibited an IC50 of 196 μg/mL. Therefore, this example shows that a combination of astaxanthin and mixed tocotrienols provided a statistically significant (p<0.05) increase in antioxidant efficacy of both astaxanthin and the mixed tocotrienols.

### EXAMPLE 2

### Antioxidant Synergy Exhibited for the Combination of β-Carotene and Mixed Tocotrienols

This example illustrates the antioxidant synergy between β-carotene and a mixture of tocotrienols when tested in the model described in Example 1. The experiment was performed as described in Example 1, except that β-carotene, obtained from Sigma (St. Louis, Mo.), was substituted for astaxanthin. Dose-response curves were described with each test article separately and then in a two-way combination. For the individual dose-response curves, concentrations of pure β-carotene used in the assay included 25.5, 51, 255 and 510 μg/mL. Following the estimation of the component IC50 values, serial dilutions of a mixture containing concentrations of each component equal to 4 times the IC50 value were assayed. This resulted in a series of test concentrations of the mixture containing 4, 2, 1, 0.5, and 0.25 times the IC50 concentration of each component.

Table 2.1 depicts the calculated inhibitory concentrations and combination indexes for 50, 75 and 90 percent inhibition of peroxy radical formation by the combination of β-carotene with mixed tocotrienols. When the mixture response was parsed into the β-carotene and tocotrienol components, values of 72, 89 and 110 μg/mL, respectively, were estimated for 50, 75 and 90 percent inhibition by β-carotene. Similarly, the tocotrienol concentrations for the same three parameters were 20, 24 and 30 μg/mL. The calculated combination index for each of the values indicated strong synergy over the complete dose-response curve.

TABLE 2.1
Statistical results of the antioxidant effect of a combination of
β-carotene and a 50% solution of mixed tocotrienols

| | IC50* | IC75* | IC90* |
|---|---|---|---|
| Test Material | [μg/mL] | [μg/mL] | [μg/mL] |
| β-Carotene alone | 196 (171-226) | | |
| 50% Tocotrienols alone | 54 (44-65) | | |
| Mixture β-Carotene | 72 (47-111) | 89 | 110 |
| Mixture Tocotrienols | 20 (13-30) | 24 (16-36) | 30 (21-44) |
| Combination Index = | 0.73 | 0.53 | 0.38 |

*Exhibited significant (p < 0.5) synergy with CI < 1.0; parenthetic values are 95% confidence intervals for the estimated value.

This combination effectively increased the antioxidant potency of both β-carotene and the tocotrienols 2.7-fold. This example shows that a combination of β-carotene and tocotrienols provided a statistically significant (p<0.05) increase in antioxidant efficacy. to 4 times the IC50 value were assayed. This resulted in a series of test concentrations of the mixture containing 4, 2, 1, 0.5, and 0.25 times the IC50 concentration of each

### EXAMPLE 3

### Antioxidant Synergy Exhibited for the Combination of Astaxanthin, Lutein and Mixed Tocotrienols

This example illustrates the antioxidant synergy for the three-way combination of astaxanthin, lutein and a mixture of tocotrienols when tested in the model described in Example 1. The experiment was performed as described in Example 1. Both astaxanthin and lutein were obtained from Sigma (St. Louis, Mo.). Dose-response curves were described with each component separately and then in a three-way combination. For the individual dose-response curves, concentrations of astaxanthin included 29, 58, 292 and 585 μg/mL; the lutein concentrations were 4, 20, 40 and 200 μg/mL and the mixed tocotrienols were tested at 12.5, 62.5, 125, 625 and 1250 μg/mL. Following the estimation of the component IC50 values, serial dilutions of a mixture containing of 4 times the IC50 of each of the three compounds was assayed. This resulted in series of test concentrations of the mixture containing 4, 2, 1, 0.5 and 0.25 times the IC50 concentration of each component.

Table 3.1 depicts the calculated inhibitory concentrations and combination indexes for 50, 75 and 90 percent inhibition of peroxy radical formation by the combination of astaxanthin and lutein with mixed tocotrienols. Values of20, 31 and 47 μg/mL, respectively, were estimated for tocotrienol responses of 50, 75 and 90 percent inhibition of free radical formation when present in the combination mixture. The calculated combination index for each of the values indicated synergy over the entire dose-response curve.

TABLE 3.1
Statistical results of the antioxidant effect of a combination of
astaxanthin, lutein and a 50 percent solution of mixed
tocotrienols

| | IC50* | IC75* | IC90* |
|---|---|---|---|
| Test Material | [μg/mL] | [μg/mL] | [μg/mL] |
| 50% Tocotrienols alone | 88 (26-298) | | |
| Astaxanthin alone | 196 (115-334) | | |
| Lutein alone | 38 (29-50) | | |
| Mixture 50% Toco- | 20 (15-26) | 31 (22-42) | 47 (32-72) |
| trienols | | | |
| Mixture Astaxanthin | 43 (33-57) | 67 | 104 |
| Mixture Lutein | 5 (4-7) | 9 | 13 |
| Combination Index = | 0.59 | 0.44 | 0.33 |

*Exhibited significant (p < 0.5) synergy with CI < 1.0; parenthetic values are 95% confidence intervals for the estimated value.

The combination of astaxanthin, lutein and tocotrienols increased the antioxidant potency of astaxanthin 4.6-fold. When tested alone, astaxanthin exhibited an IC50 of 195 μg/mL. Furthermore, in combination the concentration of the components necessary to inhibit 90 percent of the peroxy radical formation was only one-half of the concentration of the components individually required to inhibit free radical formation by 50 percent. This example shows that a combination of astaxanthin, lutein and tocotrienols provided a statistical and biological increase in antioxidant efficacy significantly greater (p<0.05) than the individual compounds over the critical regions of the dose-response curve.

### EXAMPLE 4

### Antioxidant Synergy Exhibited for the Combination of Astaxanthin, β-Carotene and Mixed Tocotrienols

This example illustrates the antioxidant synergy for the three-way combination of astaxanthin, β-carotene and a mixture of tocotrienols when tested in the model described in Example 1. The experiment was performed as described in Example 1. Both astaxanthin and β-carotene were obtained from Sigma (St. Louis, Mo.). Dose-response curves were described with each component separately and then in a three-way combination. For the individual dose-response curves, concentrations of astaxanthin included 28, 55, 111 and 222 μg/mL; the β-carotene concentrations were 45, 90, 180, 359 and 718 μg/mL and the mixed tocotrienols were tested at 12.5, 62.5, and 125 μg/mL. Following the estimation of the component IC50 values, serial dilutions of a mixture containing of 4 times the IC50 of each of the three compounds was assayed. This resulted in series of test concentrations of the mixture containing 4, 2, 1, 0.5 and 0.25 times the IC50 concentration of each component.

Table 4.1 depicts the calculated inhibitory concentrations and combination indexes for 50, 75 and 90 percent inhibition of peroxy radical formation by the combination of astaxanthin and β-carotene with mixed tocotrienols. Values of 29, 38 and 50 μg/mL, respectively, were estimated for astaxanthin responses of 50, 75 and 90 percent inhibition of free radical formation when present in the mixture with β-carotene and mixed tocotrienols. The calculated combination index for each of the values indicated synergy over the entire dose-response curve.

TABLE 4.1
Statistical results of the antioxidant effect of a combination of
astaxanthin, β-carotene and a 50 percent solution of mixed
tocotrienols

| | IC50* | IC75* | IC90* |
|---|---|---|---|
| Test Material | [μg/mL] | [μg/mL] | [μg/mL] |
| Astaxanthin alone | 182 (145-227) | | |
| β-Carotene alone | 135 (85-215) | | |
| 50% Tocotrienols alone | 54 (44-65) | | |
| Mixture Astaxanthin | 29 (17-51) | 38 (22-66) | 50 (29-89) |
| Mixture β-Carotene | 24 (13-41) | 31 | 41 |
| Mixture Tocotrienols | 11 (6-18) | 14 | 18 |
| Combination Index = | 0.53 | 0.45 | 0.40 |

*Exhibited significant (p < 0.5) synergy with CI < 1.0; parenthetic values are 95% confidence intervals for the estimated value.

The combination of astaxanthin, β-carotene and tocotrienols in the ratio of 2.7:2.2:1 increased the antioxidant potency of astaxanthin 4.6-fold. When tested alone, astaxanthin exhibited an IC50 of 195 μg/mL. Furthermore, the antioxidant activities of β-carotene and the mixed tocotrienols were increased 5.6- and 4.9-fold. This example shows that a combination of astaxanthin, β-carotene and tocotrienols provided a statistical and biological increase in antioxidant efficacy significantly greater (p<0.05) than the individual compounds over the entire dose-response curve.

### EXAMPLE 5

### Antioxidant Synergy Exhibited for the Combination of a Natural Astaxanthin Extract and Mixed Tocotrienols

This example illustrates the antioxidant synergy for the combination of a natural astaxanthin extract and a mixture of tocotrienols when tested in the model described in Example 1. The experiment was performed as described in Example 1. A natural extract containing 2.8 percent astaxanthin and a mixture of alpha-carotene, β-carotene, lutein, lycopene and zeaxanthin and adinorubin was obtained from H.Reisman Corporation, Orange, N.J. The 50 percent oil mixture of tocotrienols was also from H. Reisman Corp (Lot no. B1005-1-080799; Orange, N.J.). Within the tocotrienol fraction, approximately 56% was reported as d-gamma-tocotrienol, 30% as d-alpha-tocotrienol, 13% as d-delta-tocotrienol and 1% other tocotrienols including d-beta-tocotrienol. Dose-response curves were described with each component separately and then in a two-way combination. For the individual dose-response curves, concentrations of the astaxanthin extract included 10, 50, and 100 μg/mL and the mixed tocotrienols were tested at 12.5, 62.5, 125, 625, and 1250 μg/mL. Following the estimation of the component IC50 values, serial dilutions of a mixture containing of 4 times the IC50 of each of the three compounds was assayed. This resulted in series of test concentrations of the mixture containing 4, 2, 1, 0.5 and 0.25 times the IC50 concentration of each component.

Table 8.1 depicts the calculated inhibitory concentrations and combination indexes for 50, 75 and 90 percent inhibition of peroxy radical formation by the combination of the natural astaxanthin extract and mixed tocotrienols. Values of 7, 9 and 12 μg/mL, respectively, were estimated for astaxanthin extract responses of 50, 75 and 90 percent inhibition of free radical formation when present in the mixture with mixed tocotrineols. The calculated combination index for each of the values indicated strong synergy over the entire dose-response curve.

TABLE 8.1
Statistical results of the antioxidant effect of a combination of a natural
astaxanthin extract and a 50 percent solution of mixed tocotrienols

| | IC50* | IC75* | IC90* |
|---|---|---|---|
| Test Material | [μg/mL] | [μg/mL] | [μg/mL] |
| Astaxanthin extract alone | 71 (52-94) | | |
| 50% Tocotrienols alone | 88 (26-298) | | |
| Mixture of astaxanthin | 7 | 9 | 12 |
| extract and Tocotrienols | 17 (13-23) | 23 (17-31) | 31 (22-44) |
| Combination Index = | 0.29 | 0.17 | 0.10 |

*Exhibited significant (p < 0.5) synergy with CI < 1.0; parenthetic values are 95% confidence intervals for the estimated value.

The combination of astaxanthin extract and mixed tocotrienols in the ratio of 1:2.5 increased the antioxidant potency of the astaxanthin extract 10-fold. When tested alone, the astaxanthin extracted exhibited an IC50 of 71 μg/mL. Furthermore, the antioxidant activity of the mixed tocotrienols was increased 5-fold. This example shows that a combination consisting of a natural astaxanthin extract and mixed tocotrienols provided a statistical and biological increase in antioxidant efficacy significantly greater (p<0.05) than the individual constituent materials over the entire dose-response curve.

Thus, among the various formulations taught there has been disclosed a formulation comprising as a first component, one or more carotenoid species, and, as a second component, a tocotrienol species or derivatives thereof. These combinations provide for a synergistic antioxidant activity. It will be readily apparent to those skilled in the art that various changes and modifications of an obvious nature may be made without departing from the spirit of the invention, and all such changes and modifications are considered to fall within the scope of the invention as defined by the appended claims. Such changes and modifications would include, but not be limited to, the incipient ingredients added to affect the capsule, tablet, lotion, food or bar manufacturing process as well as vitamins, herbs, flavorings and carriers. Other such changes or modifications would include the use of other herbs or botanical products containing the combinations of the present invention disclosed above.

We claim:

1. A composition having synergistic antioxidant activity comprising an effective amount of a first component of a carotenoid species, and, as a second component, a tocotrienol species or derivatives thereof.

2. The composition of claim 1 wherein at least one of said first or second components are derived from microorganisms, plants, extracts of microorganisms or plants, or are synthetic compounds.

3. The composition of claim 1 wherein at least one of said first or second components is conjugated with a compound selected from the group consisting of mono- or di-saccharides, amino acids, fatty acids, sulfates, succinate, acetate and glutathione.

4. The composition of claim 1 additionally containing one or more members selected from the group consisting of antioxidants, vitamins, minerals proteins, fats, carbohydrates, glucosamine, chondrotin sulfate and aminosugars.

5. A composition having synergistic antioxidant activity comprising an effective amount of a first component of a carotenoid species selected from the group consisting of astaxanthin, alpha-carotene, beta-carotene, lutein, lycopene, zeaxanthin, phytoene, fucoxanthin, peridin and cantaxanthin; and as a second component, a tocotrienol species selected from the group consisting of tocotrienol, alpha-, beta-, gamma-, delta-tocotrienol, desmethyl-tocotrienol, didesmethyl-tocotrienol and mixtures thereof

6. The composition of claim 5 wherein at least one of said first or second components are derived from microorganisms, plants, extracts of microorganisms or plants, or are synthetic compounds.

7. The composition of claim 5 wherein at least one of said first or second components is conjugated with a compound selected from the group consisting of mono- or di-saccharides, amino acids, fatty acids, sulfates, succinate, acetate and glutathione.

8. The composition of claim 5 additionally containing one or more members selected from the group consisting of antioxidants, vitamins, minerals proteins, fats, carbohydrates, glucosamine, chondrotin sulfate and aminosugars.

9. A composition having synergistic antioxidant activity comprising an effective amount of a first component of a carotenoid species selected from the group consisting of astaxanthin, beta-carotene, lutein and lycopene; and as a second component, a tocotrienol species selected from the group of tocotrienol, alpha-, beta-, gamma-, delta-tocotrienol and mixtures thereof.

10. The composition of claim 9 wherein at least one of said first or second components are derived from microorganisms, plants, extracts of microorganisms or plants, or are synthetic compounds.

11. The composition of claim 9 wherein at least one of said first or second components is conjugated with a compound selected from the group consisting of mono- or di-saccharides, amino acids, fatty acids, sulfates, succinate, acetate and glutathione.

12. The composition of claim 9 additionally containing one or more members selected from the group consisting of antioxidants, vitamins, minerals proteins, fats, carbohydrates, glucosamine, chondrotin sulfate and aminosugars.

13. A composition having synergistic antioxidant activity comprising an effective amount of a first component of a pharmaceutical grade carotenoid species selected from the group consisting of astaxanthin, beta-carotene, lutein and lycopene; and as a second component, a pharmaceutical grade tocotrienol species selected from the group of tocotrienol, alpha-, beta-, gamma-, delta-tocotrienol and mixtures thereof.

14. The composition of claim 13 wherein at least one of said first or second components are derived from microorganisms, plants, extracts of microorganisms or plants, or are synthetic compounds.

15. The composition of claim 13 wherein at least one of said first or second components is conjugated with a compound selected from the group consisting of mono- or di-saccharides, amino acids, fatty acids, sulfates, succinate, acetate and glutathione.

16. The composition of claim 13 additionally containing one or more members selected from the group consisting of antioxidants, vitamins, minerals proteins, fats, carbohydrates, glucosamine, chondrotin sulfate and aminosugars.

17. A composition having synergistic antioxidant activity comprising an effective amount of a first component of a pharmaceutical grade carotenoid species selected from the group consisting of astaxanthin, beta-carotene, lutein and lycopene; and as a second component, a mixture of alpha- and beta-tocotrienol or a mixture of alpha-, beta-, gamma- and delta-tocotrienol.

18. The composition of claim 17 wherein at least one of said first or second components are derived from microorganisms, plants, extracts of microorganisms or plants, or are synthetic compounds.

19. The composition of claim 17 wherein at least one of said first or second components is conjugated with a compound selected from the group consisting of mono- or di-saccharides, amino acids, fatty acids, sulfates, succinate, acetate and glutathione.

20. The composition of claim 17 additionally containing one or more members selected from the group consisting of antioxidants, vitamins, minerals proteins, fats, carbohydrates, glucosamine, chondrotin sulfate and aminosugars.

21. A composition having synergistic antioxidant activity comprising an effective amount of a first component of a pharmaceutical grade carotenoid species selected from the group consisting of astaxanthin, beta-carotene, lutein and lycopene; and as a second component, a mixture of alpha-, beta-, gamma- and delta-tocotrienol.

22. The composition of claim 21 wherein at least one of said first or second components are derived from microorganisms, plants, extracts of microorganisms or plants, or are synthetic compounds.

23. The composition of claim 21 wherein at least one of said first or second components is conjugated with a compound selected from the group consisting of mono- or di-saccharides, amino acids, fatty acids, sulfates, succinate, acetate and glutathione.

24. The composition of claim 21 additionally containing one or more members selected from the group consisting of antioxidants, vitamins, minerals proteins, fats, carbohydrates, glucosamine, chondrotin sulfate and aminosugars.

25. A method for normalization and therapeutic treatment of symptoms of oxidative stress in warm blooded animals comprising administering to an animal a composition comprising an effective amount of a first component of a carotenoid species, and as a second component, a tocotrienol species and derivatives thereof; and continuing said administration until said symptoms of oxidative stress are reduced.

26. The method of claim 25 wherein the composition is formulated in a dosage form such that said administration provides 0.1 to 50 mg/day of a cartenoid species, and 0.1 to 1200 mg/day of a tocotrienol species.

27. The method of claim 25, wherein the composition is administered in an amount sufficient to maintain a serum or tissue concentration of 0.001 to 500 μM of a cartenoid species, and 0.008 to 500 μM of a tocotrienol species.

29. The method of claim 25 wherein the first component and the second component are administered in a sequential manner or in a substantially simultaneous manner.

30. A method for normalization and therapeutic treatment of symptoms of oxidative stress in warm blooded animals comprising administering to an animal a composition comprising an effective amount of a first component of a carotenoid species selected from the group consisting of astaxanthin, alpha-carotene, beta-carotene, lutein, lycopene, zeaxanthin, phytoene, fucoxanthin, peridin and cantaxanthin; and as a second component, a tocotrienol species selected from the group consisting of tocotrienol, alpha-, beta-, gamma-, delta-tocotrienol, desmethyl-tocotrienol, didesmethyl-tocotrienol and mixtures thereof; and continuing said administration until said symptoms of oxidative stress are reduced.

31. The method of claim 30 wherein the composition is formulated in a dosage form such that said administration provides 0.1 to 50 mg/day of a cartenoid species, and 0.1 to 1200 mg/day of a tocotrienol species.

32. The method of claim 30 wherein the composition is administered in an amount sufficient to maintain a serum or tissue concentration of 0.001 to 500 μM of a cartenoid species, and 0.008 to 500 μM of a tocotrienol species.

33. The method of claim 30 wherein the first component and the second component are administered in a sequential manner or in a substantially simultaneous manner.

34. A method for normalization and therapeutic treatment of symptoms of oxidative stress in warm blooded animals comprising administering to an animal a composition comprising an effective amount of a first component of a carotenoid species selected from the group consisting of astaxanthin, beta-carotene, lutein and lycopene; and as a second component, a tocotrienol species selected from the group of tocotrienol, alpha-, beta-, gamma-, delta-tocotrienol and mixtures thereof; and continuing said administration until said symptoms of oxidative stress are reduced.

35. The method of claim 34 wherein the composition is formulated in a dosage form such that said administration provides 0.1 to 50 mg/day of a cartenoid species, and 0.1 to 1200 mg/day of a tocotrienol species.

36. The method of claim 34, wherein the composition is administered in an amount sufficient to maintain a serum or tissue concentration of 0.001 to 500 μM of a cartenoid species, and 0.008 to 500 μM of a tocotrienol species.

37. The method of claim 34 wherein the first component and the second component are administered in a sequential manner or in a substantially simultaneous manner.

38. A method for normalization and therapeutic treatment of symptoms of oxidative stress in warm blooded animals comprising administering to an animal a composition comprising an effective amount of a first component of a pharmaceutical grade carotenoid species selected from the group consisting of astaxanthin, beta-carotene, lutein and lycopene; and as a second component, a pharmaceutical grade tocotrienol species selected from the group of tocotrienol, alpha-, beta-, gamma-, delta-tocotrienol and mixtures thereof; and continuing said administration until said symptoms of oxidative stress are reduced.

39. The method of claim 38 wherein the composition is formulated in a dosage form such that said administration provides 0.1 to 50 mg/day of a cartenoid species, and 0.1 to 1200 mg/day of a tocotrienol species.

40. The method of claim 38, wherein the composition is administered in an amount sufficient to maintain a serum or tissue concentration of 0.001 to 500 μM of a cartenoid species, and 0.008 to 500 μM of a tocotrienol species.

41. The method of claim 38 wherein the first component and the second component are administered in a sequential manner or in a substantially simultaneous manner.
